(19) United States
(12) Patent Application Publication
(10) Pub. No.: US 2007/0167477 A1
(43) Pub. Date: Jul. 19, 2007

(54) PROCESSES TO PREPARE FINASTERIDE POLYMORPHS

(76) Inventors: Venkata Naga Brahmeswara Rao Mandava, Hyderabad 500 072, Andhra Pradesh (IN); Radhakrishna Singamsetty, Hyderabad 500 072. Andhra Pradesh (IN); Nagaraju Manne, Nalgonda 508 277, Andhra Pradesh. (IN); Satish Kumar Vujjini, Hyderabad 500 070, Andhra Pradesh (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/622,555

(22) Filed: Jan. 12, 2007

Related U.S. Application Data
(60) Provisional application No. 60/747,973, filed on May 23, 2006.

(30) Foreign Application Priority Data
Jan. 13, 2006 (IN) 57/CHE/2006

(51) Int. Cl. A61K 31/44

(52) U.S. Cl. 514/290

(57) ABSTRACT

Processes for preparing polymorphic crystalline Form I and Form III of finasteride.

### INTRODUCTION TO THE INVENTION

The present invention relates to processes for the preparation of finasteride crystalline polymorphic Form I and Form III.

Finasteride is chemically known as 4-azaandrost-1-ene-17-carboxamide,N-(1,1-dimethylethyl)-3-oxo-,(5α,17β) and has the structural Formula I.

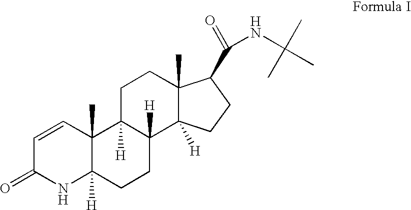

Finasteride is a synthetic 4-azasteroid compound useful as a specific inhibitor of steroid Type II 5α-reductase, an intracellular enzyme that converts the androgen testosterone into 5α-dihydrotestosterone (DHT). It is commercially available in the form of oral tablets under the brand names PROPECIA™ (1 mg), for the treatment of male pattern hair loss, and PROSCAR™ (5 mg), for the treatment of symptomatic benign prostatic hyperplasia (BPH) in men.

U.S. Pat. No. 4,760,071 discloses finasteride, a process for its preparation, pharmaceutical compositions comprising finasteride and their method of use.

U.S. Pat. Nos. 5,652,365 and 5,886,184 disclose crystalline Form I and Form II of finasteride and processes for their preparation.

U.S. Pat. No. 6,677,453 discloses a process for the preparation of crystalline Form I of finasteride by forming a complex with Group (I) and Group (II) metal salts.

International Application Publication No. 2004/039828 discloses a process for the preparation of crystalline Form I of finasteride by slow cooling of a finasteride solution in substantially anhydrous alkyl acetate solvents.

International Application Publication No. 2005/003149 A1 discloses a process for the preparation of crystalline Form I of finasteride by dissolving finasteride in a solvent and replacing the solvent partially or completely with an anti-solvent using distillation.

International Application Publication No. 2002/20553 discloses crystalline Form III, Form IV and Form V of finasteride and processes for their preparation.

The aforesaid processes for the preparation of Form I and Form III involve multiple step operations, hazardous solvents and give inconsistent results in terms of polymorph purity and yield.

There is a continuing need to prepare crystalline forms of active substances such as finasteride in an industrially simple and readily feasible way with high yields.

### SUMMARY OF THE INVENTION

The present invention relates to processes for the preparation of finasteride crystalline Form I and Form III.

In one aspect, the present invention relates to a process for the preparation of crystalline Form I of finasteride comprising the steps of:
a) providing a solution of finasteride in an alcohol;
b) combining the solution of step a) with water to precipitate a solid; and
c) recovering the crystalline Form I of finasteride.

In another aspect, the present invention provides a process for the preparation of crystalline Form III of finasteride comprising the steps of:
a) providing a solution of finasteride in a suitable solvent;
b) combining the solution of step a) with cyclohexane to precipitate a solid; and
c) recovering the crystalline Form III of finasteride.

In an embodiment of the invention, a process for preparing crystalline finasteride Form I comprises combining an alcohol solution of finasteride with water.

In another embodiment of the invention, a process for preparing crystalline finasteride Form III comprises combining a solution of finasteride in an organic solvent with cyclohexane.

Finasteride Form I and Form III obtained by the processes of the present invention are characterized by their X-ray powder diffraction patterns.

The processes of the present invention are simple, cost effective, eco-friendly, reproducible and industrially scalable. Moreover, finasteride Form I and Form III obtained by the processes of the present invention are free flowing and well suited for use in preparing pharmaceutical formulations.

### BRIEF DESCRIPTION OF THE DRAWINGS

FIG. 1 is an X-ray powder diffraction (XRPD) pattern of finasteride crystalline Form I prepared according to Example 3.

FIG. 2 is an XRPD pattern of finasteride crystalline Form III prepared according to Example 5.

### DETAILED DESCRIPTION OF THE INVENTION

The present invention relates to processes for the preparation of finasteride crystalline Form I and Form III.

In one aspect, the present invention relates to a process for the preparation of crystalline Form I of finasteride comprising the steps of:
a) providing a solution of finasteride in an alcohol;
b) combining the solution of step a) with water to precipitate a solid; and
c) recovering the crystalline Form I of finasteride.

The step a) of providing a solution of finasteride in an alcohol includes dissolving finasteride in an alcohol or obtaining such a solution from a previous processing step wherein finasteride is formed.

When the solution is prepared by dissolving finasteride in a suitable solvent, any form of finasteride such as any crystalline or amorphous form, including any salts, solvates and hydrates, may be utilized for preparing the solution.

Suitable alcohols that can be used for providing solution of finasteride include but are not limited to methanol, ethanol, isopropanol, n-butanol, isobutanol, tertiary-butyl alcohol, secondary butyl alcohol and the like or mixtures thereof.

The concentration of the solute can range from about 50 g/l to about 350 g/l in the solvent. Any other concentration may be used as long as a clear solution is obtained, and the maximum solubility will, of course, differ according to the particular solvent that is being used and the temperature of the solution.

Suitable temperatures for providing the solution of finasteride may range form about 0 to 100° C., or about 20 to 40° C. Any temperature can be used to obtain a clear solution as long as it does not affect the purity of the finasteride.

The solution obtained in step a) can be optionally treated with activated charcoal to enhance the color of the compound followed by filtration through an inert medium such as a flux calcinated diatomaceous earth (Hyflow) bed to remove the carbon, or by using other clarification techniques known to those skilled in the art.

Depending upon the equipment used and the concentration and temperature of the solution, the filtration apparatus may need to be preheated to avoid premature crystallization.

Step b) involves combining the solution obtained in step a) with water.

Suitably combining the finasteride solution with water can be done by adding water to the finasteride solution or by adding finasteride solution to the water.

Optionally, small amounts of seeding crystals of finasteride Form I may be added to the mixture. Small amounts such as about 0.1 to 5 weight %, or about 0.2 to 0.5 weight %, of the water are usually used. Seeding crystals may be added before, or where appropriate, after initiating the precipitation.

Suitable temperatures for combing the finasteride solution with water range from about 20° C. to about 70° C.

The amount of water that can be used can range from about 1 to about 5 times the volume of the solution, or about 1.5 to about 2.5 times the volume of the solution.

To enhance the crystallization, the mixture may be further maintained for about 1 to 10 hours, or longer, at temperatures such as, for example, about 10° C. to about 25° C. The exact temperature and time required for complete crystallization can be readily determined by a person skilled in the art and will also depend on parameters such as concentration and temperature of the solution or slurry.

Step c) involves recovering the crystalline Form I of finasteride, and can involve using any technique such as filtration by gravity or by suction, centrifugation, decantation, and the like.

The wet solid thus obtained after separation can be dried using any technique such as fluid bed drying (FBD), spin flash drying, aerial drying, or oven drying, under atmospheric or reduced pressure. For example, drying can be performed under reduced pressure or under atmospheric pressure at a temperature of at about 40° C. to 120° C., or 80° C. to 90° C., depending on the volatility of the solvent that is being removed. The atmosphere for drying can be air or a partially or completely inert atmosphere, such as by using nitrogen.

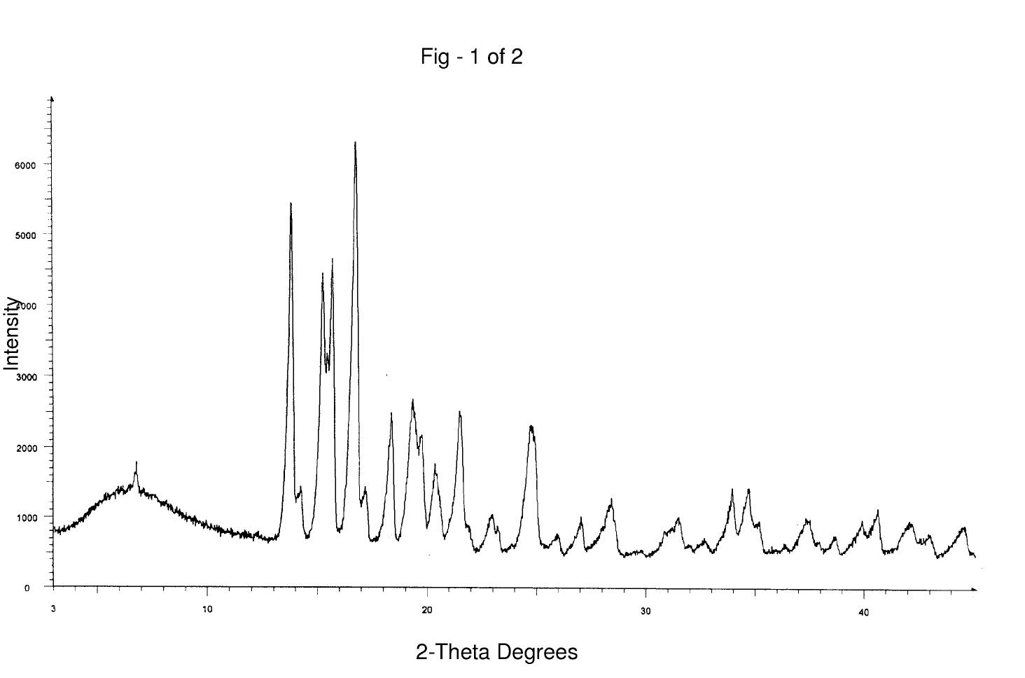

Crystalline Form I of finasteride obtained by the process of present invention is characterized by its X-ray powder diffraction pattern substantially in accordance with FIG. 1. Crystalline Form I of finasteride obtained by the process of present invention is characterized by its X-ray powder diffraction pattern having major peaks at about 13.8, 15.6, 16.7, 18.3, 19.3, 20.3, 22.9, 24.7, and 28.4, ±0.2 degrees 2 theta.

Crystalline Form I of finasteride obtained by the process of present invention is further characterized by its differential scanning calorimetry (DSC) curve. The DSC curve has been generated at a heating rate of 20° C./minute and in a closed cup, exhibiting a minor endotherm with a peak temperature of about 232° C.; an extrapolated onset temperature of about 223° C. with an associated heat of about 11 joules/g, and a major melting endotherm with a peak temperature of about 261° C., an extrapolated onset temperature of about 258° C. with an associated heat of about 89 J/g.

In another aspect, the present invention provides a process for the preparation of crystalline Form III of finasteride comprising the steps of:
a) providing a solution of finasteride in a suitable solvent;
b) combining the solution of step a) with cyclohexane to precipitate a solid; and
c) recovering the crystalline Form III of finasteride.

The step a) of providing a solution of finasteride in a suitable solvent includes dissolving finasteride in a suitable solvent or obtaining such a solution form a previous processing step wherein finasteride is formed.

When the solution is prepared by dissolving finasteride in a suitable solvent, any form of finasteride such as any crystalline or amorphous form, including any salts, solvates and hydrates may be utilized for preparing the solution.

Suitable solvents that can be used for providing solution of finasteride include but are not limited to halogenated hydrocarbons such dichloromethane, chloroform, carbon tetrachloride, ethylene dichloride and the like; aromatic hydrocarbons such as toluene, xylene and the like; alkyl acetates such as ethyl acetate, propyl acetate and the like; and mixtures thereof.

The concentration of the solute can range from about 50 g/l to about 200 g/l of the solution. Any other concentration may be used as long as a clear solution is obtained, and the maximum solubility will, of course, differ according to the particular solvent that is being used and the temperature of the solution.

Suitable temperatures for providing the solution of finasteride may range form about 0 to 100° C., or about 20 to 40° C. Any temperature can be used to obtain a clear solution as long as it does not affect the purity of finasteride.

The solution obtained in step a) can be optionally treated with activated charcoal to enhance the color of the compound followed by filtration through an inert medium such as flux calcinated diatomaceous earth (Hyflow) bed to remove the carbon or by using other clarification techniques known to those skilled in the art.

Depending upon the equipment used and the concentration and temperature of the solution, the filtration apparatus may need to be preheated to avoid premature crystallization.

Step b) involves combining the solution of step a) with cyclohexane to precipitate a solid.

Suitably combining the finasteride solution with cyclohexane can be done by adding cyclohexane to the finasteride solution or by adding finasteride solution to the cyclohexane.

Optionally, small amounts of seeding crystals of finasteride Form III may be added to the mixture. Small amounts such as about 0.1 to 5 weight %, or about 0.2 to 0.5 weight %, of the finasteride in solution are used. Seeding crystals may be added before, or where appropriate, after initiating the precipitation.

Suitable temperatures for combining the finasteride solution with cyclohexane range from about 20° C. to about 70° C.

The amount of cyclohexane that can be used ranges from about 1 to about 5 times the volume of the solution, or about 1.5 to about 2.5 times the volume of the solution.

Suitably, a part of the solvent may be distilled off from the mixture to increase the concentration of finasteride in the mixture.

To enhance the crystallization, reaction mixture may be further maintained for about 1 to 10 hours, or longer, at temperatures such as for example about 10° C. to about 25° C. The exact temperature and time required for complete crystallization can be readily determined by a person skilled in the art and will also depend on parameters such as concentration and temperature of the solution or slurry.

Step c) involves recovering the crystalline Form III of finasteride, such as using any technique such as filtration by gravity or by suction, centrifugation, decantation, and the like.

The wet solid thus obtained after filtration can be dried using any technique such as fluid bed drying (FBD), spin flash drying, aerial drying, or oven drying under ambient or reduced pressure. For example, drying can be performed under reduced pressure or under atmospheric pressure at a temperature of at about 40° C. to 120° C., or 80° C. to 90° C., depending on the volatility of the solvent that is being removed. The atmosphere for drying can be air or a partially or completely inert atmosphere, such as by using nitrogen.

Crystalline Form III of finasteride obtained by the process of the present invention is characterized by its X-ray powder diffraction pattern substantially in accordance with FIG. 3. Crystalline Form III of finasteride obtained by the process of the present invention is characterized by its X-ray powder diffraction pattern having major peaks at about 5.2, 10.6, 13.5, 14.8, 15.8, 16.0, 16.4, 17.1, 18.1, and 19.44, ±0.2 degrees 2 theta.

Crystalline Form III of finasteride obtained by the process of present invention is further characterized by its differential scanning calorimetry (DSC) curve. A DSC curve has been generated using a heating rate of 10° C./minute and in a closed cup, exhibiting a major melting endotherm with a peak temperature of about 259° C., an extrapolated onset temperature of about 257° C. with an associated heat of about 86.5 joules/g.

All the XRPD patterns described herein were measured on Bruker Axe, D8 Advance Powder X-ray Diffractometer with a Cu K alpha-1 radiation source.

The finasteride crystalline Form I and Form III of the present invention are stable, free flowing and well suited for use in preparing stable pharmaceutical formulations.

The processes of the present invention are a simple, efficient, cost effective, ecofriendly, reproducible, scalable and robust process to produce the desired finasteride crystalline Form I and Form III.

These and other specific aspects and embodiments of this invention are described in further detail by the examples below, which examples are provided only for illustration and are not intended to limit the scope of the appended claims in any manner.

### EXAMPLE 1

Preparation of 17-β-(N-tert, butyl carbamoyl-)-3-oxo-4-aza-5-α-androstane:

400 L of dichloromethane, 40 kg of 3 oxo-4-aza-5-alpha-androstane-17-Beta-carboxylic acid, 29.5 kg of dicyclohexylcarbodiimide, and 27.5 kg of 1-hydroxy benzotriazole were taken into a reactor and the reaction mass was heated to 40° C. the reaction mass was maintained under reflux for 5 hours. Reaction completion was checked using thin layer chromatography. After the reaction was completed, the reaction mass was cooled to 17° C. and filtered. The filter bed was washed with 160 L of dichloromethane. 35 Kg of tertiary-butylamine was taken into another reactor and the above filtrate was added to it. The reaction mass was maintained at 30° C. for 4 hours. Reaction completion was checked using thin layer chromatography. After the reaction was completed, the reaction mass was cooled to 18° C. and filtered. The filter bed was washed with 320 L of dichloromethane. The combined filtrate was washed with a solution of 20 L of hydrochloric acid in 380 L of water. The combined filtrate was then washed with 800 L of water in two equal lots. The filtrate was taken into another reactor and distilled until the remaining volume of the concentrate is approximately 150 L. The concentrate was cooled to 18° C. and filtered through a Hyflow bed and the bed was washed with 10 L of dichloromethane. The filtrate was taken into another reactor and distilled completely below 45° C. 80 L of ethyl acetate was added to the residue and again distilled completely below 75° C. under a vacuum of 450 mm Hg. The remaining residue was cooled to 30° C. and 400 L of ethyl acetate was added to it. The reaction mass was heated to 50° C. and stirred for 20 minutes. The reaction mass was cooled to 30° C. and maintained for 40 minutes. The separated solid was filtered and the filtered bed was washed with 110 L of ethyl acetate. The wet material was dried at 65° C. for 3 hours. The dried material was taken into another reactor and 102 L of methanol was added to it. The reaction mass was stirred for 2 hours for clear dissolution. 257 L of water was added to the above solution maintained at 30° C. for 1 hour. The separated solid was filtered and washed with 68.4 L of water. The wet solid was dried at 65° C. for 6 hours to yield 33.9 Kg of the title compound.

### EXAMPLE 2

Preparation of Finasteride

Charged 100 g of 17-β-(N-tert, butyl carbamoyl-)-3-oxo-4-aza-5-α-androstane, 80.1 g of 2,3-Dichloro-5,6-dicyanobenzoquinone, 278 ml of N,O-Bis(trimethylsilyl) trifluoroacetamide, and 2 L of toluene into a round bottom flask under a nitrogen atmosphere. The reaction mass was heated to 93° C. and maintained for 6 hours. A solution of 1116 g of sodium sulfite in 11.16 L of water was prepared and heated to 55° C. The reaction mass was washed with 2170 ml of hot sodium sulfite solution prepared above. The reaction mass was then washed with hot sodium sulfite solution using nine lots of 1 L each. 18 L of water was taken into another round bottom flask and heated to 70° C. The reaction mass was washed with hot water prepared above in six equal lots. The organic layer was separated and distilled completely under a vacuum of 300 mm Hg at 65° C. To the residue, 150 ml of ethyl acetate and 150 ml of tetrahydrofuran were added and heated to 45° C. The reaction mass was maintained at 45° C. for 30 minutes. The reaction mass was then allowed to cool, and 3.72 ml of water was added to it. The reaction mass was cooled to −3° C. and maintained for one hour. The separated solid was filtered and washed with 44 ml of an equal volume mixture of ethyl acetate and tetrahydrofuran.

The wet solid was taken into another round bottom flask and 176.4 ml of acetic acid was added to it. 264.6 ml of water was added. 25.94 ml of methanol was added to the reaction mass and heating given to 72° C. The reaction mass was maintained at 72° C. for 45 minutes and then cooled to 32° C. The reaction mass was maintained at 32° C. for 10 hours and 50.4 ml of water was added to it. The reaction mass was further cooled to 12° C. and maintained for 20 minutes. The separated solid was filtered and washed with 1500 ml of water to yield 66.8 g of the wet title compound.

### EXAMPLE 3

Preparation of Finasteride Crystalline Form I

42 g of finasteride and 150 ml of methanol were charged to a clean and dry round bottom flask followed by stirring for about 5 minutes at about 30° C. to obtain a homogenous solution. 5 g of activated charcoal was charged and stirred for about 5 minutes. The resultant reaction suspension was filtered through a Hyflow bed and the Hyflow bed was washed with methanol (2×75 ml). 600 ml of water was charged into a clean and dry round bottom flask at 30° C. The above obtained methanolic solution was added slowly to water over about 10 minutes. The resultant suspension was stirred for about 50 minutes at 30° C. The solid was filtered and washed with 100 ml of water. The wet solid obtained was dried at about 80° C. for about 6 hours to afford 27 g of the desired title compound.

Water content by Karl Fischer (KF): 0.11% w/w.

### EXAMPLE 4

Preparation of Finasteride Crystalline Form I

120 L of methanol and 20.5 kg of wet finasteride were taken into a reactor and heated to 45° C. to obtain a clear solution. 2.5 kg of carbon was added to the solution and stirred for 5 minutes. The mass was filtered and the filter bed was washed with 50 L of methanol. The combined filtrate was taken into another reactor and distilled until only 20% of the original volume remained in the reactor, then was cooled to 30° C. 120 L of water was taken into another reactor and the above concentrated filtrate was added and stirred for 2 hours. The separated solid was filtered and washed with 100 L of water. The wet solid was dried at 84° C. for 24 hours. The dried solid was sifted to get 10.1 Kg of the title compound.

### EXAMPLE 5

Preparation of Finasteride Crystalline Form III

40 L of ethyl acetate, 21.08 kg of crude finasteride and 4 L of water were charged into a clean and dry reactor followed by heating to about 50-55° C. The resultant suspension was agitated for about 30 minutes at about 50-55° C. and then cooled to about −5 to 5° C. for 60 minutes. The solid was separated by centrifugation followed by washing the solid with 7 L of ethyl acetate. The wet solid was charged into a clean and dry reactor containing 160 L of toluene. The resultant mixture was heated to about 80-90° C. and stirred for about 20 minutes to obtain a clear homogenous solution. The solution was cooled to about 25-35° C. followed by stirring for about 1.5 hours. The solid that separated was isolated in a centrifuge and washed with 11 L of toluene.

120 L of toluene was charged into a clean and dry reactor containing the wet solid obtained above. The resultant mass was heated to about 80-90° C. and stirred for about 20 minutes to obtain a clear homogenous solution. The solution was cooled to about 25-35° C. followed by stirring for about 1.5 hours. The resultant solid was isolated by centrifuging and washed with 11 L of toluene.

85 L of toluene was charged into a clean and dry reactor containing the wet solid obtained. The resultant mass was heated to about 80-90° C. and stirred for about 20 minutes to obtain a clear homogenous solution. The solution was cooled to about 25-35° C. and stirred for about 1.5 hours followed by centrifuging to separate the solid. The solid was washed with 8 L of toluene.

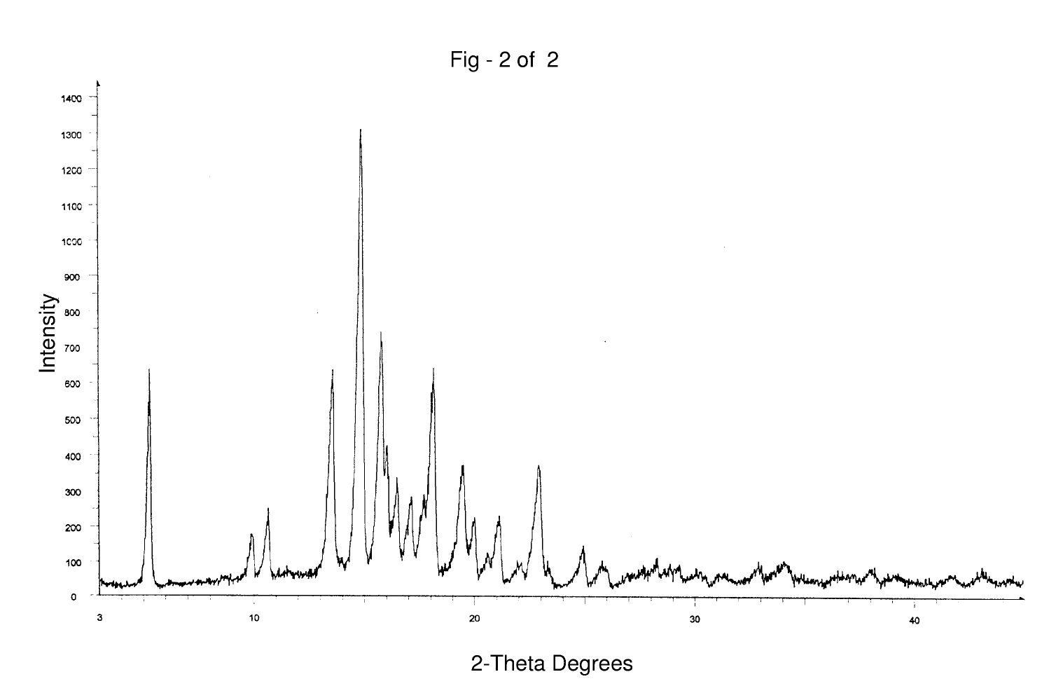

6.5 kg of wet finasteride obtained above was charged into a reactor containing 65 L of dichloromethane and stirred at 25-30° C. for about 15 minutes to get a clear solution. 400 g of activated charcoal was added to the above solution and stirred at 25-30° C. for about 30 minutes. The resultant suspension was filtered through a Hyflow bed and the Hyflow bed was washed with 70 L of dichloromethane. Total filtrate was concentrated to about 18 L at about 35-40° C. under vacuum. 500 L of cyclohexane was charged into a clean and dry reactor and 75 g of crystalline Form III of finasteride was added. The above obtained concentrated dichloromethane solution was added to the cyclohexane at about 25-35° C. and agitated for about 2 hours. 135 L of the solvent was distilled from the suspension at about 80° C. and the resultant residual mass was refluxed at about 80-90° C. for about 4 hours. The mixture was cooled to about 25-35° C. and the separated solid was separated in a centrifuge followed by washing with 20 L of cyclohexane. The wet solid thus obtained was dried at about 80-90° C. for about 40 hours to yield 4.75 kg of crystalline Form III of finasteride having an X-ray powder diffraction pattern in accordance with FIG. 2.

### EXAMPLE 6

Preparation of Finasteride Crystalline Form III

50 g of finasteride was charged into clean dry flask containing 812.5 ml of dichloromethane and stirred at 25-30° C. for about 15 minutes to get a clear solution. 5 g of activated charcoal was added to the above solution and stirred at 25-30° C. for about 30 minutes. The resultant suspension was filtered through a Hyflow bed and the Hyflow bed was washed with 875 ml of dichloromethane. Total filtrate was concentrated to about 250 ml at about 28° C. under vacuum. 2 L of cyclohexane was taken into another clean and dry flask and 1.0 g of crystalline Form III of finasteride was added. The above obtained concentrated dichloromethane solution was charged to the cyclohexane at about 25-35° C. under agitation. 1670 ml of the solvent was distilled off from the suspension at about 80° C., the residual mass was cooled to about 25-35° C. and the separated solid was filtered followed by washing with 100 ml of cyclohexane. The wet solid thus obtained was dried at about 93° C. for about 12 hours to yield 49.2 g of crystalline Form III of finasteride.

1. A process for preparing crystalline finasteride Form I, comprising combining an alcohol solution of finasteride with water.

2. The process of claim 1, wherein an alcohol comprises methanol.

3. The process of claim 1, wherein a solution comprises about 50 to about 350 g/L finasteride.

4. The process of claim 1, wherein an amount of water is about 1 to about 5 times a volume of solution.

5. The process of claim 1, wherein an amount of water is about 1.5 to about 2.5 times a volume of solution.

6. A process for preparing crystalline finasteride Form III, comprising combining a solution of finasteride in an organic solvent with cyclohexane.

7. The process of claim 6, wherein an organic solvent comprises a halogenated hydrocarbon, an aromatic hydrocarbon, an alkyl acetate, or a mixture thereof.

8. The process of claim 6, wherein an organic solvent comprises a halogenated hydrocarbon.

9. The process of claim 6, wherein an organic solvent comprises dichloromethane.

10. The process of claim 6, wherein a solution comprises about 50 to about 200 g/L finasteride.

11. The process of claim 6, wherein an amount of cyclohexane is about 1 to about 5 times a volume of solution.

12. The process of claim 6, wherein an amount of cyclohexane is about 1.5 to about 2.5 times a volume of solution.
